(19) United States
(12) Patent Application Publication
(10) Pub. No.: US 2008/0131442 A1
(43) Pub. Date: Jun. 5, 2008

(54) IgY antibodies to human telomerase reverse transcriptase

(75) Inventor: Yan Xiao, Clarksburg, MD (US)

Correspondence Address: BANNER & WITCOFF, LTD., 1100 13th STREET, N.W., SUITE 1200, WASHINGTON, DC, 20005-4051, US

(73) Assignee: SCIENCE APPLICATIONS INTERNATIONAL CORPORATION, San Diego, CA (US)

(21) Appl. No.: 11/474,452

(22) Filed: Jun. 26, 2006

(51) Int. Cl. A61K 39/395; G01N 33/53; C07K 16/40

(52) U.S. Cl. 424/146.1; 435/7.1; 530/388.26; 977/902

(57) ABSTRACT

IgY antibodies which specifically bind to human telomerase reverse transcriptase (hTERT) can be used to detect hTERT and thereby diagnose cancer. Preparations of the antibodies can be used to reduce hTERT reverse transcriptase activity in in vitro assays and can be used as in vivo therapeutics to treat cancer.

The U.S. Government has certain rights in this invention as provided for by the terms of Naval Research Laboratory contract no. N00173-03-2013.

The application file contains at least one drawing executed in color. Copies of this patent application publication with color drawings will be provided by the Office upon request and payment of the necessary fee.

### FIELD OF THE INVENTION

The invention relates to IgY antibodies to human telomerase reverse transcriptase (hTERT) and their therapeutic and diagnostic uses.

### BACKGROUND OF THE INVENTION

Telomere length is determined by telomerase, an RNA-dependent DNA polymerase. When active, telomerase lengthens one chain of the telomere repeating sequence and maintains the length of the telomere. Human telomerase is a complex which comprises two structural units: human telomerase RNA (hTR) and human telomerase reverse transcriptase (hTERT). hTR is an RNA molecule that forms the template for extending a strand of the telomere DNA. hTERT is an enzyme subunit that catalyzes the template-dependent polymerization reaction.

In most human cells, the length of the telomere shortens with each division. The telomeres of cancer cells, however, do not shorten even with repeated cell division. Cancer cells, unlike most normal cells, express telomerase (Shay et al., Eur. J. Cancer 33, 787, 1997). The expression of telomerase activity in cancer cells is regulated by hTERT (Nakamura et al., Science 277, 955, 1997; Nakayama et al., Nature Genetics 18, 65, 1998). Thus, agents which can detect hTERT are useful diagnostics, and agents which can reduce hTERT reverse transcriptase activity are useful therapeutics. Agents which have both functions would be particularly useful.

### SUMMARY OF THE INVENTION

One embodiment of the invention is a preparation comprising an IgY antibody which specifically binds to human telomerase reverse transcriptase (hTERT). The antibody can be, for example, a polyclonal antibody, a monoclonal antibody, an Fab fragment, an F(ab′)₂ fragment, a single chain antibody, a minibody, a diabody, a Kappa body, or a Janusin. Some IgY antibodies specifically bind, for example, to an hTERT epitope comprising the amino acid sequence SEQ ID NO:2. Other antibodies specifically bind to an hTERT epitope comprising the amino acid sequence SEQ ID NO:3. The preparation can comprise a pharmaceutically acceptable vehicle.

Another embodiment of the invention is a method of detecting hTERT. A biological sample is contacted with an IgY antibody which specifically binds to hTERT. Binding of the IgY antibody to hTERT is detected. The biological sample can comprise cancer cells. The antibody can be, for example, a polyclonal antibody, a monoclonal antibody, an Fab fragment, an F(ab′)₂ fragment, a single chain antibody, a minibody, a diabody, a Kappa body, or a Janusin. Some IgY antibodies specifically bind, for example, to an hTERT epitope comprising the amino acid sequence SEQ ID NO:2. Other antibodies specifically bind to an hTERT epitope comprising the amino acid sequence SEQ ID NO:3. Various means can be used to detect binding of the IgY antibody to hTERT, such as a quantum dot detection system.

Yet another embodiment of the invention is a method of reducing hTERT reverse transcriptase activity. hTERT is contacted with an IgY antibody which specifically binds to hTERT, thereby reducing hTERT reverse transcriptase activity. The hTERT can be in a cell, and cell can be in vitro or in vivo. The cell can be in a patient who has a cancer. The antibody can be, for example, a polyclonal antibody, a monoclonal antibody, an Fab fragment, an F(ab′)₂ fragment, a single chain antibody, a minibody, a diabody, a Kappa body, or a Janusin. Some IgY antibodies specifically bind, for example, to an hTERT epitope comprising the amino acid sequence SEQ ID NO:2. Other antibodies specifically bind to an hTERT epitope comprising the amino acid sequence SEQ ID NO:3.

### BRIEF DESCRIPTION OF THE FIGURES

FIG. 1. Photomicrographs showing immunohistochemical detection of hTERT by chicken polyclonal IgY antibodies and mammalian IgG antibodies. FIG. 1A, Human telomerase detection with IgY antibodies in high telomerase-expressing (A549, left) and control cells (IMR-90, right). FIG. 1B, Human telomerase detection with mammalian IgG antibodies in high telomerase-expressing cells (A549, left) and cells that express telomerase at very low levels (IMR-90, right).

FIG. 2. Deconvolution image processing planes (top four panels, left and right) of cells for telomerase expression in high telomerase-expressing cells (A549, left stack) and very low-telomerase expressing cells (IMR-90, right stack). The total in-focus fluorescence is represented on the bottom grids for high telomerase expressing cells (A549, left bottom) and control cells (IMR-90, right bottom). These are the same cell images as in FIG. 1A.

FIG. 3. Bar graph showing results of quantum dot imaging of antibody-hTERT binding using IgY and IgG antibodies in high telomerase expressing cells (A549) and control cells (IMR-90).

FIG. 4. Telomerase in Western blots as detected by IgM (top panel), IgG (second from top), and IgY (third from top). The lowest panel shows the β-actin control. See Example 5.

FIG. 5. Histogram comparing the relative affinity for hTERT of three antibodies (IgY, IgG, IgM) in a layered peptide array assay (see Example 7).

### DETAILED DESCRIPTION OF THE INVENTION

The invention provides immunoglobulin Y (IgY) antibodies which specifically bind to human telomerase reverse transcriptase (hTERT). “IgY antibody” as used herein includes intact polyclonal or monoclonal IgY molecules; IgY fragments, such as monomeric and dimeric Fab, F(ab′)₂, scFv, and Fv; and non-naturally occurring molecules such as diabodies, minibodies, Kappa bodies, Janusins, and the like. Naturally occurring IgY antibodies are produced in egg-laying non-mammalian vertebrates (e.g., chickens, ducks, geese, snakes, urodele amphibians).

IgY antibodies provide significant advantages when compared with previously generated mammalian antibodies with specificity for hTERT. In general, IgY antibodies do not react with rheumafactors, human Fc receptors, bacterial Fc receptors, or human anti-mouse IgG antibodies, which makes them very safe for therapeutic use. IgY antibodies of the invention in particular are surprisingly more sensitive than commercially available mammalian hTERT antibodies and can be used, inter alia, in early cancer diagnostic test platforms and as therapeutic agents for treating cancer and benign proliferative diseases in which hTERT is expressed.

IgY antibodies of the invention comprise an hTERT binding site and specifically bind to hTERT. “hTERT binding sites” as used herein include hTERT binding sites which naturally occur in the variable portion of IgY antibodies. hTERT binding sites also include binding sites which differ from naturally occurring hTERT binding sites by between 1 and 15 (e.g., 1, 2, 3, 4, 5, 6, 7, 8, 9, 10, 11, 12, 13, 14, or 15) conservative amino acid substitutions and which specifically bind to hTERT. Typically, an IgY antibody which specifically binds to hTERT provides a detection signal at least 5-, 10-, or 20-fold higher than a detection signal provided with a non-hTERT human antigen when used in an immunochemical assay. Preferably, IgY antibodies which specifically bind to hTERT do not detect other human proteins in immunochemical assays and can immunoprecipitate hTERT from solution. Preferred IgY antibodies of the invention reduce hTERT reverse transcriptase activity (e.g., by 10, 20, 25, 30, 40, 50, 60, 70, 75, 80, 90, or 100%). Enzyme activity can be measured as described in Soldateschi et al., J. Biotechnol. 118, 370-78, 2005

High affinity IgY antibodies are preferred, such as those which bind to hTERT with at least 1-, 2-, 3-, 4-, 5-, 6-, 7-, 8-, 9-, or 10-fold higher affinity than an IgG or IgM hTERT antibody. The affinity of IgY antibody binding to hTERT can be assayed using any method known in the art, including technologies such as layered peptide arrays (see Example 7) and surface plasmon resonance (Day et al., Protein Science 11, 1017-25, 2002; Sjolander & Urbaniczky, Anal. Chem. 63, 2338-45, 1991; Szabo et al., Curr. Opin. Struct. Biol. 5, 699-705, 1995).

### Preparation of IgY Antibodies

Various methods can be used to prepare and collect IgY antibodies, depending on the form of the antibody desired. See, e.g., Gee et al., US 2002/0028917; Larsson et al., U.S. Pat. No. 6,537,550; Suzuki & Lee, Glycobiol. 14, 275-92, 2004; Miyamoto et al., Biologicals, Epub Mar. 28, 2006; Malik et al., J. Vet. Sci. 7, 43-46, 2006; Ruan et al., Protein Expr Purif. 44, 45-51 (Epub Apr. 2, 2005); Schade et al., Altern. Lab. Anim. 33, 129-54, 2005; Kazimierczuk et al., Acta Biochim. Pol. 52, 261-66, 2005; Hassl, J. Immunol. Methods. 297, 253-57, Epub Jan. 11, 2005; Boxio et al., J. Appl. Physiol. 98, 905-10, 2005. Epub Nov. 5, 2004; Shin et al., J. Vet. Sci. 3, 47-57, 2002; Tsen et al., Clin. Chem. 49, 810-13, 2003; Lobbedey & Schlatterer, J. Vet. Med. B Infect. Dis. Vet. Public Health. 50, 81-85, 2003; Carlander et al., Ups. J. Med. Sci. 104, 179-89, 1999; Kummer & Li-Chan, J. Immunol. Methods. 211, 125-37, 1998; Bando & Higgins, Immunology 89, 8-12, 1996; Schade & Hlinak, ALTEX 13, 5-9, 1996; Polson, U.S. Pat. No. 4,550,019; Polson et al., Immunol. Commun. 9, 475-93, 1980; and Polson et al., Immunol. Commun. 9, 495-514, 1980.

Polyclonal IgY antibodies of the invention can be produced in egg-laying non-mammalian mammalian vertebrates, such as fowl (e.g., chickens, ducks, geese), snakes, or urodele amphibians (e.g., axolotls, newts), after immunizing the animal with an hTERT epitope or a nucleic acid molecule encoding an hTERT epitope (see, e.g., Romito et al., Biotechniques September;31(3):670, 672, 674-75, 2001). Preferred hTERT epitopes include amino acids 165-348 (SEQ ID NO:2 ) and amino acids 227-338 (SEQ ID NO:3). Amino acids 165-348 are exposed on the outer surface of hTERT and, compared with the rest of the hTERT protein, have lower homology with TERT proteins of other species. Full-length hTERT (SEQ ID NO:1) also can be used as an immunogen. One method of producing IgY polyclonal antibodies in chickens is described in Example 1, below.

Monoclonal IgY antibodies (e.g., full-length, scFv, Fv) can be prepared using any technique which provides for the production of antibody molecules by continuous cell lines in culture. These techniques include, but are not limited to, the hybridoma technique, the human B-cell hybridoma technique, and the EBV-hybridoma technique. See Roberge et al., Science 269, 202-204, 1995; Kohler et al., Nature 256, 495-497, 1985; Kozbor et al., J. Immunol. Methods 81, 31-42, 1985; Cote et al., Proc. Natl. Acad. Sci. 80, 2026-2030, 1983; and Shimamoto et al., Biologicals, September 2005; 33(3):169-74. Single chain antibodies can be generated by chain shuffling from random combinatorial IgY libraries. Takeda et al., Nature 314, 452-454, 1985.

Single-chain antibodies also can be constructed using a DNA amplification method, such as PCR, using hybridoma cDNA as a template. Single-chain antibodies can be mono- or bispecific, and can be bivalent or tetravalent. Construction of tetravalent, bispecific single-chain antibodies is well known in the art. A nucleotide sequence encoding a single-chain antibody can be constructed using manual or automated nucleotide synthesis, cloned into an expression construct using standard recombinant DNA methods, and introduced into a cell to express the coding sequence. Alternatively, single-chain antibodies can be produced directly using, for example, filamentous phage technology. Burton et al., Proc. Natl. Acad. Sci. 88, 11120-23, 1991; Verhaar et al., Int. J Cancer 61, 497-501, 1995.

IgY antibodies of the invention can be purified from any cell which expresses the antibodies, including host cells which have been transfected with IgY antibody-encoding nucleic acid molecules. The host cells are cultured under conditions suitable for expression of the IgY antibodies. Appropriate host cells and culture conditions can be selected from the wide variety known in the art.

Purified IgY antibodies are separated from other compounds that normally associate with the antibody in the cell, such as certain proteins, carbohydrates, or lipids. Purification methods include, but are not limited to, size exclusion chromatography, ammonium sulfate fractionation, ion exchange chromatography, affinity chromatography, and preparative gel electrophoresis. A preparation of purified IgY antibodies is at least 80% pure; preferably, the preparations are 90%, 95%, or 99% pure. Purity of the preparations can be assessed by any means known in the art, such as SDS-polyacrylamide gel electrophoresis. A preparation of purified IgY antibodies of the invention can contain more than one type of IgY antibody which specifically binds to hTERT.

Full-length IgY polyclonal or monoclonal antibodies, however prepared, can be cleaved with standard techniques to obtain functional antibody fragments such as Fab or F(ab′)₂. See Cheung et al., Protein Expr. Purif 32, 135-40, 2003. Binding proteins which are derived from immunoglobulins and which are multivalent and multispecific, such as the “diabodies” described in WO 94/13804 and Holliger et al., Proc. Natl. Acad. Sci. USA 90, 6444-48, 1993; the “minibodies” described in Martin et al., EMBO J. 13, 5303-09, 1994; “Kappa bodies” described in Ill et al., Protein Eng. 10, 949-57, 1997; and “Janusins” (bispecific single chain molecules) described in Traunecker et al., EMBO J. 10, 3655 3659, 1991, and Traunecker et al., Int. J. Cancer Suppl. 7, 51-52, 1992, can be prepared.

Any IgY antibody of the invention also can be produced using chemical methods to synthesize its amino acid sequence, such as by direct peptide synthesis using solid-phase techniques (Merrifield, J. Am. Chem. Soc. 85, 2149-54, 1963; Roberge et al., Science 269, 202-04, 1995). Protein synthesis can be performed using manual techniques or by automation. Automated synthesis can be achieved, for example, using Applied Biosystems 431A Peptide Synthesizer (Perkin Elmer). Optionally, fragments of IgY antibodies can be separately synthesized and combined using chemical methods to produce a full-length molecule. The newly synthesized molecules can be substantially purified by preparative high performance liquid chromatography (e.g., Creighton, PROTEINS: STRUCTURES AND MOLECULAR PRINCIPLES, WH Freeman and Co., New York, N.Y., 1983). The composition of a synthetic polypeptide can be confirmed by amino acid analysis or sequencing (e.g., using Edman degradation).

Detection of hTERT

IgY antibodies of the invention can be used diagnostically, to detect telomerase-expressing cells in a biological sample. Such biological samples include, but are not limited to, samples of blood or other body fluid (e.g., body fluid urine, sputum, etc.) or solid tissue (e.g., surgical biopsies, forensic samples), cell lines, and primary cell cultures.

Any detection methods known in the art can be used to detect binding of IgY antibodies to hTERT. These methods include, but are not limited to, Western blots, ELISAs, radioimmunoassays, immunohistochemical assays, immunoprecipitation, and other immunochemical assays known in the art. Detection can be qualitative or quantitative. In some embodiments, binding of IgY antibodies to hTERT is detected using streptavidin-conjugated semiconductor nanocrystals as described in Example 4. Use of such “quantum dot” detection systems readily permits quantitative immunohistochemistry staining.

Preferred methods of the invention include detection and quantification of hTERT-antibody binding using quantum dot detection systems; z-plane fluorescence image capture using three dimensional-deconvolution microscopy (e.g., Chamgoulov & MacAulay, Cell Oncol. 26, 319-27, 2004; Hollars & Dunn, Biophys. J. 75, 342-53, 1998); high-throughput, automated robotic slide processing; and affinity estimates using quantitative, massively parallel, high-throughput analysis of peptide antigen-antibody interactions using a layered peptide array (LPA) technology (Gannot et al., J. Mol. Diagn. 7, 427-36, 2005). The working examples below describe immunohistochemistry (IHC) and LPA methods. These examples illustrate a general platform imaging approach to quantitative comparisons of antibody-based cancer biomarkers.

Methods of detecting hTERT using IgY antibodies according to the invention provide more information than, for example, telomere repeat amplification protocol (TRAP) and RT-PCR assays, in which cell structure is destroyed. In addition, assays of the invention can detect telomerase expressed in very small cell populations, including in a single cell.

### Therapeutic Agents

Preferred IgY antibodies of the invention reduce hTERT reverse transcriptase activity and can be provided in a pharmaceutical composition to treat cancer and other proliferative diseases in which hTERT is expressed in a mammal, preferably a human. If desired, an IgY antibody of the invention can be conjugated to a T-cell epitope, a toxin, or a radionuclide-binding peptide or protein to bring a killing function close to the cancer cells.

Cancers which can be treated include, but are not limited to blood cancers (e.g., leukemias, lymphomas) and cancers of solid tissues (e.g., bladder, bone, brain, breast, cervix, colon, esophagus, kidney, liver, lung, pancreas, prostate, stomach).

Those skilled in the art can use known injectable, physiologically acceptable sterile solutions to prepare suitable pharmaceutical compositions comprising IgY antibodies of the invention. Aqueous isotonic solutions, such as saline or corresponding plasma protein solutions, are readily available and can be used to prepare ready-to-use solutions for parenteral injection or infusion. Pharmaceutical compositions can be stored as lyophylisates or dry preparations, which can be reconstituted with a known injectable solution before use. A pharmaceutical composition can be supplemented with known carrier substances or/and additives (e.g., serun albumin, dextrose, sodium bisulfite, EDTA, etc.). Pharmaceutical compositions of the invention typically comprise a pharmaceutically acceptable vehicle, such as an inert diluent.

Pharmaceutical compositions of the invention can be administered by different routes known to those skilled in the art. For systemic application, the intravenous, intravascular, intramuscular, intraarterial, intraperitoneal, oral, intranodal, or intrathecal routes can be used. More localized application can be effected subcutaneously, intracutaneously, intracardially, intralobally, intramedullarly, intrapulmonarily, or directly in or near the tissue to be treated.

Data obtained from cell culture assays and animal studies can be used to formulate a range of doses for use in humans. Many cancer cell lines of various types are available for use in in vitro assays to assess the effects of IgY antibodies of the invention on properties of cancer cells, including proliferation rate, apoptosis, invasiveness (for example, into soft agar), cellular morphology, etc. Cancer cell lines can be obtained, for example, from the American Type Culture Collection (ATCC), P.O. Box 1549, Manassas, Va. 20108.

Animal models for cancer are known and widely used in the art. See, e.g., U.S. Pat. No. 6,706,947 (immunosuppressed animals carrying a tumor xenograft); U.S. Pat. No. 6,750,206 (nude (BALBC nu/nu) mice with implanted subcutaneous tumors); Lupu et al., J Gastrointest Surg. May 2006; 10(5): 635-45 (mouse colon cancer model); Sun et al., World J Gastroenterol. May 7, 2006; 12(17): 2785-8 (transgenic mouse model of pancreatic cancer); Blouin et al., Clin Exp Metastasis. e-pub May 3, 2006 (rat models of bone cancer); Hong et al., Clin Cancer Res. Apr. 15, 2006; 12(8): 2563-7 (rabbit model of liver cancer); and Savai et al., J Immunother. March-April 2006; 29(2):175-87 (mouse model of lung cancer).

Depending on the desired duration and effectiveness of the treatment, compositions may be administered once or several times, for example on a daily basis for several days, weeks or months, and in different dosages. Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Antibodies which exhibit large therapeutic indices are preferred. Appropriate doses preferably lie within a range of circulating antibody concentrations which includes the ED₅₀ with little or no toxicity. The dosage may vary within this range depending upon the dosage form employed and the route of administration utilized. Doses can range from 1 ng/kg to 100 mg/kg and will depend on age, condition, sex and extent of the disease in the patient. For example, doses can vary from 5 μg/kg to about 50 μg/kg, about 50 μg/kg to about 5 mg/kg, about 100 μg/kg to about 500 μg/kg, and about 200 μg/kg to about 250 μg/kg of patient body weight.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLE 1

### Generation of Chicken Polyclonal IgY Antibodies

A portion of human telomerase (GenBank No. NP_{—}003210.1; SEQ ID NO:1), amino acids 165-348 (SEQ ID NO:2), was expressed as a recombinant polypeptide, purified from E. coli cells, and used as an immunogen. The immunogen was injected intramuscularly into laying hens (leghom and Rhode Island). About 200 μg of protein was mixed with Complete Freund's Adjuvant (CFA) for primary immunization. Booster inoculations were performed at 2-3 weeks interval with 100 μg antigen in Incomplete Freund's Adjuvant (IFA) for three inoculation cycles. Antibodies were isolated from eggs collected after a second boost with polyethylene glycol (PEG) (Polson et al., Immunol. Commun. 9, 475-93, 1980). Eggs were collected after second boost. Antibodies were isolated from egg yolk using polyethylene glycol (PEG) precipitation method (Polson et al., Immunol. Commun. 9, 475-93, 1980). The antibodies were purified by affinity chromatography using the polypeptide immunogen as the affinity ligand.

### EXAMPLE 2

### Immunohistochemistry

Two cell lines were used. A549 cells have high telomerase levels. IMR-90 cells have low telomerase levels. Telomerase activity and transcript levels in these cell lines were determined by TRAP and RT-PCR assays, respectively (Atha et al., Electrophoresis. 24, 109-14, 2003; Jakupciak et al., Clinical Chemistry 51, 1-9, 2005).

Cells were grown on tissue culture chamber slides (LabTek No. 177429) in medium recommended by the supplier at a cell density of 30,000 cells/cm². Cell monolayers were fixed in neutral-buffered zinc formalin (10%, Fisher 313-095).

Immunohistochemistry substrates were prepared by two methods. In one method, fixed monolayers were pre-blocked with 5% low-fat dry milk in PBS, 20° C., for 20 minutes, slides were incubated manually with primary antibody (1:300 dilution), biotin-labeled secondary antibody (1:300 dilution), and detection reagents (streptavidin-Qdot605, Qdot Corporation/Invitrogen). In the other method (high-throughput), slides were prepared robotically (primary, secondary antibodies, and washes) with a Ventana BENCHMARK® XT workstation (Ventana, Tucson, Ariz.) (Bankfalvi et al., Int J Oncol. 25, 929-3, 2004).

IgY antibodies were detected by biotin-labeled anti-IgY antibodies followed by semiconductor nanocrystal fluorophore streptavidin conjugates (Qdot Corporation). The imaging system for three-dimensional analysis of fluorescence signals from quantum dots, deconvolution of z-plane (depth) images, and integration of the signal are described in Xiao et al., Nature Methods 2, 723, 2005. Semiconductor nanocrystal fluorophores (quantum dots 605) signals were visualized on an Olympus BH2 RCFA microscope equipped with Qdot605 filters (Chroma Technology, Inc., E460SPUV exciter, 475CXRU dichroic, D605/40 emitter). Three-dimensional images were captured under identical conditions (exposure time=150 ms) with a Quantix CCD camera (Photometrics, Inc., Tucson, Ariz.) through IPLabs software (version 3.6, Signal Analytics, Inc.); the images were deconvolved using Huygens 2 (Scientific Volume Imaging); quantitation deconvolved three-dimensional images using Imaris 4 (Bitplane).

### Western Blotting

Cell lysates were prepared as described in Xiao et al., Clinical Cancer Research 10, 8683-86, 2004. Electrophoresis was carried out using pre-cast polyacrylarnide/SDS gels (NUPAGE® 4-12% Bis-Tris gels; Invitrogen No. NP0321BOX) at 100 volts for 1-3 hrs. Gels were equilibrated in buffer and electroblotted onto nitrocellulose membranes. Transfer blots were soaked in blocking buffer containing 5% w/v nonfat dry milk in 1× PBS and 0.1% v/v TWEEN 20®. The blots were incubated with primary antibodies (1:1000 dilution) overnight at 4° C. at antibody concentrations of 0.5, 1, 2, 4, 8, and 16 μg/ml. The blots were then washed and incubated with horseradish peroxidase-conjugated secondary antibodies (1:1000 dilution; goat anti-chicken IgY, GenWay Biotech, Inc., No. GAYFC-HRP; or goat anti-mouse IgG+IgM, Abcam, No. ab6006) for 1 hour at room temperature. After washing with TWEEN®-PBS, bound antibody was visualized by SIGMAFAST™ DAB tablet sets (No. D-4293).

### EXAMPLE 3

### Layered Peptide Array (LPA) Analysis

Layered peptide array methods were carried out as described in Gannot et al., J. Molec. Diagnostics 7, 427-36, 2005. P-FILM antibody affinity membranes (20/20 GeneSystems, Inc., Rockville, Md.) coated with the hTERT polypeptide were placed within a vacuum plate (Bio-Rad, California). Antibodies were applied to the 96 wells in the plate in duplicate and incubated for 5 minutes. Vacuum was applied for 5 minutes followed by washing of the membranes for 5 minutes in TBST, which is 50 mM Tris-HCl (Quality Biological Inc., Maryland), 150 mM NaCl (KD Medical, Maryland), and 150 mM TWEEN 20® (Bio-Rad, California). Incubation with secondary antibodies (FITC- or Alexa-conjugated) was carried out for 30 minutes at room temperature with shaking, followed by another wash in TBST. Membranes were dried on a filter paper (Whatman, N.J. USA) and scanned on a Typhoon scanner with 520 BP40 and ALEXA filters (Typhoon 9410, Amersham Biosciences, New Jersey).

### LPA Density Measurements and Statistical Analysis

Images of the membranes were imported to IMAGEPRO® 4.5 analysis software (MediaCybernetics, Maryland) for analysis. Each membrane included 96 dots (one for each well). A circle was marked around each dot, and the software calculated the optical density according to the formula [OD=−Log 10(x/256)], with 256 representing the total number of gray levels in the image and X the individual level of gray of each object (dot). Each measurement was repeated twice. This analysis generated a data set of average optical densities for each antibody in the membranes. The data were imported to Microsoft EXCEL®, and the mean ± standard deviation values were calculated.

### EXAMPLE 4

Comparison of Chicken IgY Polyclonal hTERT Antibodies With Commercial hTERT Antibodies

Several antibodies were used to detect telomerase in A549 cells (high telomerase expression) and IMR-90 cells (controls). Mouse anti-human telomerase IgM antibodies were from Novus Biologicals (NB100-297,); the antigen was full-length recombinant human telomerase protein produced in insect cells. IgG2a was obtained from Novocastra (NCL-hTERT); the antigen was a synthetic peptide corresponding to residues 173-320 near the N-terminus of human telomerase. Antibody details are summarized below:

| Antibody/Isotype | Type |
|---|---|
| YXPB-IgY-hTel/p | chicken IgY polyclonal (Example 1) |
| NCL-hTERT | murine monoclonal (Novocastra) |
| (IgG₂ₐ) | |
| NB100-297 (IgM) | murine monoclonal (Novus Biologicals) |

Although both the mammalian IgGs and chicken IgYs were detected with the same secondary fluorophores (streptavidin/Qdot605 conjugates), the detection antibodies (anti-IgG or anti-IgM, and anti-IgY) were different, so only relative comparisons were made among the antibody isotypes.

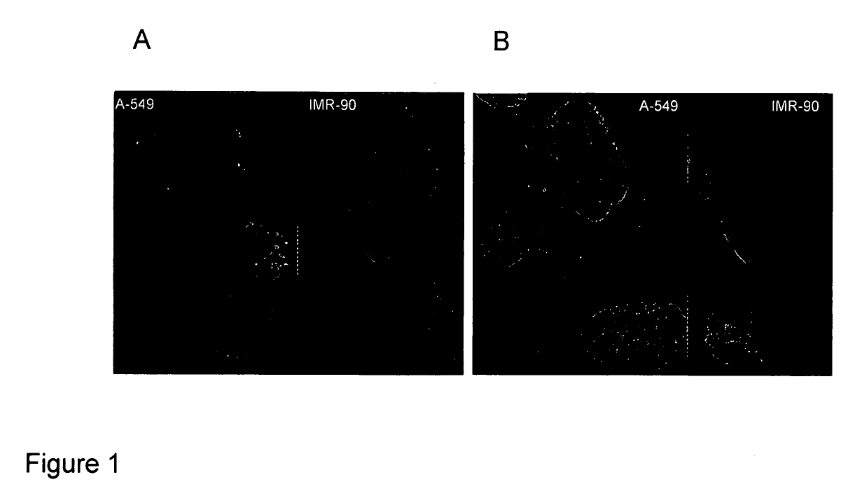

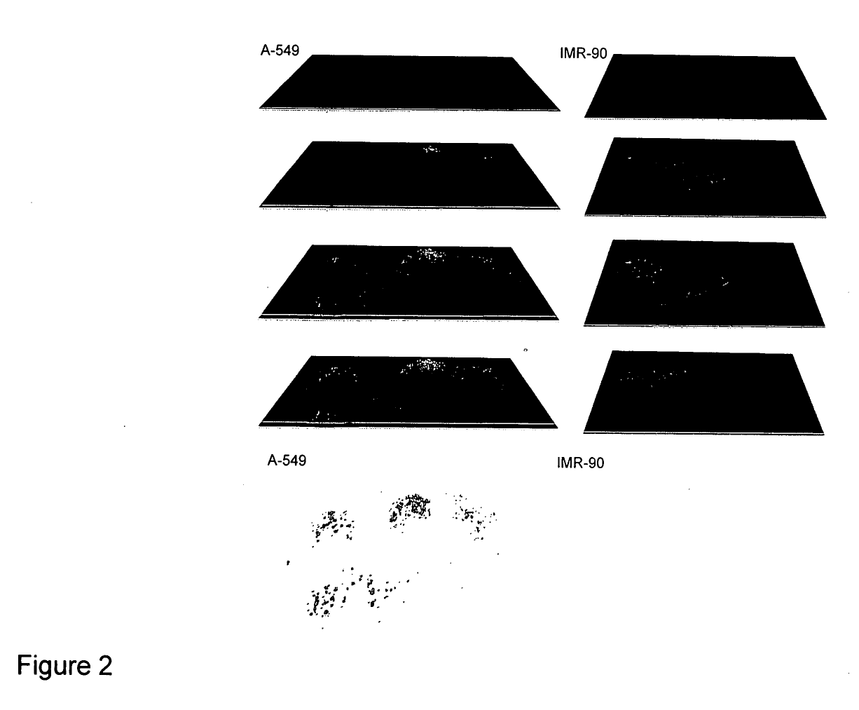

The polyclonal IgY antibodies detected epitopes exclusively within the cell nucleus in high telomerase-expressing A549 cells (FIG. 1A, left) but not in the control IMR-90 cells (FIG. 1A, right). Deconvolution of separate representative stack images of telomerase signals in A549 cells (left) and IMR-90 cells (right) is shown in FIG. 2.

Cumulative stack fluorescence is shown on the orientation grids in the lowermost panels of FIG. 2. The nuclear signal in the A549 cells was heterogeneous. Under similar conditions, the mammalian IgG (FIG. 1B, left and right) detected a much lower signal in A549 cells. The telomerase-specific IgM elicited almost no detectable signal in A549 cells.

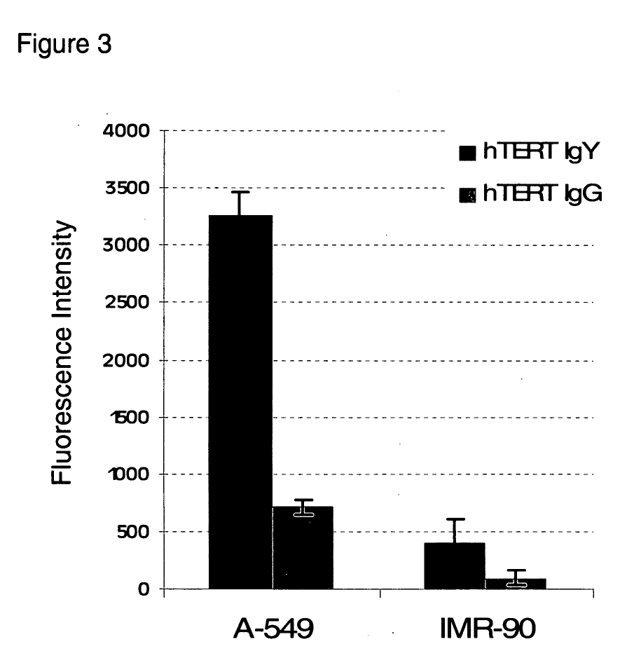

Measurements of total Qdot605 fluorescence in ten cells are shown quantitatively in FIG. 3. The chick anti-telomerase IgY antibodies provided about a 4-fold greater signal than the mammalian IgG anti-telomerase antibodies (FIG. 3 right panel, 3269 vs 712 fluorescence units).

### EXAMPLE 5

### Features of Molecular Targets Detected by IgYs

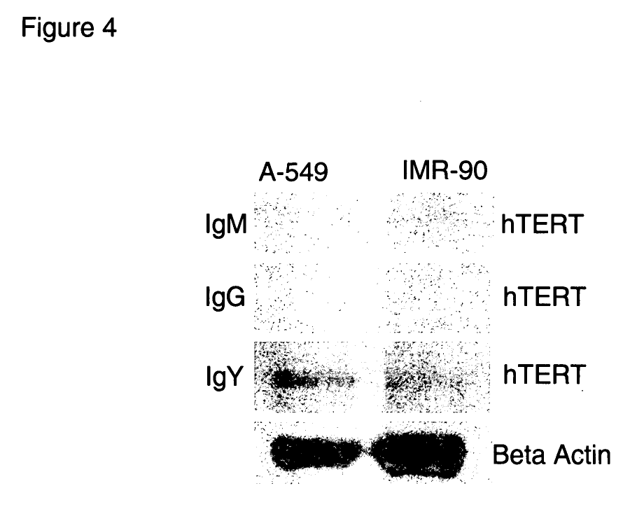

Western blots were made to determine whether the antibodies tested in Example 4 detected the appropriate sized-target proteins in telomerase expressing cells, but not in control cells. As a control, chicken IgY isotype antibody ab13822 specific for human beta-actin was obtained (Abcam) and used at a 1:1000 dilution. The results are shown in FIG. 4.

Approximately equal amounts of protein lysate were loaded in each lane (FIG. 4, bottom panel, left and right). The IgM anti-telomerase antibody showed no signal and no difference between telomerase expressing (A549 cells) and control IMR-90 cells (FIG. 4, top panel, left and right). The anti-telomerase IgG detected at most a faint 127 kD band for telomerase in A549 cells (FIG. 4, second panel left); this finding demonstrates that the IgG antibody is not useful for Western blot detection of telomerase. In contrast, the chicken IgY anti-telomerase antibodies detected a 127 kD band in A549 cells; this band was absent in IMR-90 cells (FIG. 4, third panel from top, left and right respectively).

The detected band was the correct molecular weight for telomerase. These data, taken with the immunohistochemistry data in Example 4, demonstrate that the IgY antibodies detected the appropriate target sites in telomerase-expressing, but not in control cells.

### EXAMPLE 7

Quantitative Comparisons of Chicken Polyclonal hTERT IgY Antibodies With Commercial Antibodies

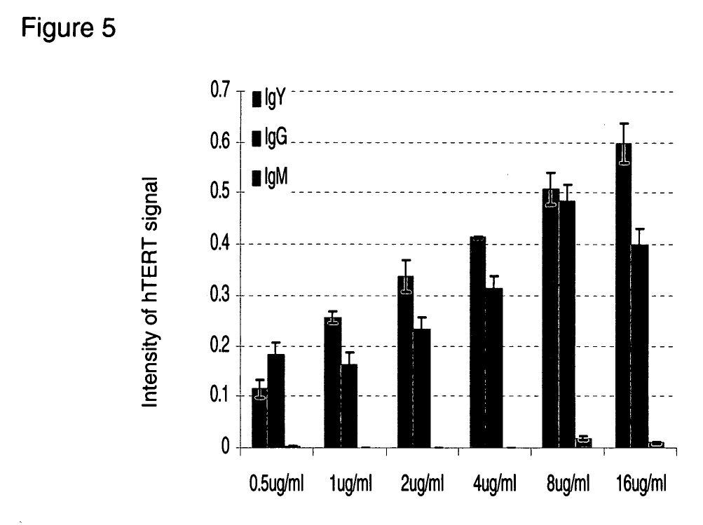

A preliminary estimate of antibody affinity for telomerase was made by testing antibodies against a dilution series of the hTERT polypeptide antigens using layered peptide array (LPA) technology (Gannot et al., 2005). In such experiments, antibodies are tested at varying concentrations for affinity to a polypeptide antigen covalently bound to an assay membrane. The results are shown in FIG. 5.

At lower concentrations (0.5 μg/ml), the affinity of the mammalian monoclonal IgG antibody for telomerase was about 30% greater than the affinity of the polyclonal IgY antibodies. However, at all antibody concentrations tested from 1 μg/mL through 16 μg/mL, the IgY antibodies provided a significantly better signal except at 8 μg/mL (at this concentration, the IgG and IgY signal had overlapping standard errors). In most cases, the IgY signal was about 30% greater than the signal obtained with the IgG antibody. In this experiment, consistent with immunohistochemistry data, the IgM signal was weak or absent.

1. A preparation comprising an IgY antibody which specifically binds to human telomerase reverse transcriptase (hTERT).

2. The preparation of claim 1 wherein the antibody is a polyclonal antibody.

3. The preparation of claim 1 wherein the antibody is a monoclonal antibody.

4. The preparation of claim 1 wherein the antibody is selected from the group consisting of an Fab fragment, an F(ab′)₂ fragment, a single chain antibody, a minibody, a diabody, a Kappa body, and a Janusin.

5. The preparation of claim 1 wherein the antibody specifically binds to an hTERT epitope comprising the amino acid sequence SEQ ID NO:2.

6. The preparation of claim 1 wherein the antibody specifically binds to an hTERT epitope comprising the amino acid sequence SEQ ID NO:3.

7. The preparation of claim 1 further comprising a pharmaceutically acceptable vehicle.

8. A method of detecting hTERT, comprising:
contacting a biological sample with an IgY antibody which specifically binds to hTERT; and
detecting binding of the IgY antibody to hTERT.

9. The method of claim 8 wherein the antibody is a polyclonal antibody.

10. The method of claim 8 wherein the antibody is a monoclonal antibody.

11. The method of claim 8 wherein the antibody is selected from the group consisting of an Fab fragment, an F(ab′)₂ fragment, a single chain antibody, a minibody, a diabody, a Kappa body, and a Janusin.

12. The method of claim 8 wherein the antibody specifically binds to an hTERT epitope comprising the amino acid sequence SEQ ID NO:2.

13. The method of claim 8 wherein the antibody specifically binds to an hTERT epitope comprising the amino acid sequence SEQ ID NO:3.

14. The method of claim 8 wherein the biological sample comprises cancer cells.

15. The method of claim 8 wherein binding of the IgY antibody to hTERT is detected using a quantum dot detection system.

16. A method of reducing hTERT reverse transcriptase activity, comprising:
contacting hTERT with an IgY antibody which specifically binds to hTERT, whereby hTERT reverse transcriptase activity is reduced.

17. The method of claim 16 wherein the antibody is a polyclonal antibody.

18. The method of claim 16 wherein the antibody is a monoclonal antibody.

19. The method of claim 16 wherein the antibody is selected from the group consisting of an Fab fragment, an F(ab′)₂ fragment, a single chain antibody, a minibody, a diabody, a Kappa body, and a Janusin.

20. The method of claim 16 wherein the antibody specifically binds to an hTERT epitope comprising the amino acid sequence SEQ ID NO:2.

21. The method of claim 16 wherein the antibody specifically binds to an hTERT epitope comprising the amino acid sequence SEQ ID NO:3.

22. The method of claim 16 wherein the hTERT is in a cell.

23. The method of claim 22 wherein the cell is in vitro.

24. The method of claim 22 wherein the cell is in vivo.

25. The method of claim 22 wherein the cell is in a patient who has a cancer.


SEQUENCE LISTING (3 sequences)

SEQ ID NO: 1 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 2 - protein, 184 aa, Homo sapiens
SCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLS

SEQ ID NO: 3 - protein, 112 aa, Homo sapiens
SASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDK